(19) United States
(12) Patent Application Publication
(10) Pub. No.: US 2006/0224124 A1
(43) Pub. Date: Oct. 5, 2006

(54) Administering device for an injectable product with a release lock

(76) Inventor: Benjamin Scherer, Uster (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/373,524

(22) Filed: Mar. 10, 2006

Related U.S. Application Data
(63) Continuation of application No. PCT/CH04/00564, filed on Sep. 8, 2004.

(30) Foreign Application Priority Data
Sep. 11, 2003 (DE) 103 42 058.4

(51) Int. Cl. A61M 5/20; A61M 5/315

(52) U.S. Cl. 604/220; 604/135

(57) ABSTRACT

A device for administering an injectable product includes a drive unit for driving a drive member in an administering direction, a release mechanism for releasing the drive unit and a locking mechanism which, when locked, prevents the release mechanism from being operated, wherein the locking mechanism is displaceable generally along the longitudinal axis of the device from a locked to an unlocked position and the release mechanism can be moved generally radially with respect to the longitudinal axis for releasing purposes when the locking mechanism is in the unlocked position.

### CROSS-REFERENCE TO RELATED APPLICATION(S)

This application is a continuation of International Application No. PCT/CH2004/000564, filed on Sep. 8, 2004, which claims priority to German Application No. 103 42 058.4, filed on Sep. 11, 2003, the entire contents of which are incorporated by reference herein.

### BACKGROUND

The present invention relates to devices and methods for delivering, administering, injecting or dispensing substances, and to methods of making and using such devices. More particularly, it relates to a device for administering an injectable product, more particularly an automatic injection device, with a drive unit for driving a drive member, a release mechanism for releasing the drive unit and a locking mechanism which, when locked, prevents the release mechanism from being operated to release the drive unit.

There are numerous models of administering devices for injectable products, mostly fluid products such as insulin, growth hormone, etc. The injectable product is generally accommodated in an ampoule inside a housing of the device or in a chamber in the housing, and can be administered or delivered from the device by means of a dispensing mechanism. To make the administering procedure more simple for a doctor or for a patient in the case of self-administration, automatic injections devices are used. Generally, in such devices an injection needle accommodated in the housing of the device pierces a surface, such as human tissue by means of an automatic mechanism and the injectable product is then administered immediately, also automatically, by a dispensing mechanism. Also known are needleless administering devices, by means of which the injectable product is discharged from the device on the basis of a selected pressure curve so that it penetrates a tissue surface.

In order to prevent an administering device from being undesirably or inadvertently released, various locking mechanisms are known which ensure that the administering device is not released. U.S. Pat. No. 6,135,979, for example, discloses an administering device with a drug cartridge for a fluid product and a compressed gas spring which serves as the drive unit for administering the product. The drug cartridge and the gas spring are accommodated in a housing comprising a first housing part incorporating the cartridge, an outlet for the product and a closure cap, and a second housing part incorporating the gas spring, the second housing part being displaceable towards the first housing part in the direction in which the product is administered. A drive rod adjoins the gas spring and is retained in a pre-tensioned locked state by means of a pin which locates in a groove in the rod. In the locked state, the two housing parts are disposed at a distance apart from one another and the pin abuts with an internal face of the second housing part so that it can not release the rod. For locking purposes, the two housing parts are held at a distance apart by a strip, which is disposed in the gap between the housing parts. In order to administer the injectable product, the strip is tom off so that the second housing part can be moved along the longitudinal axis of the device in the administering direction when the administering device is pressed, e.g., on a tissue surface. As a result, the second housing part moves relative to the gas spring, the rod and the pin so that an orifice in the second housing part moves so that it sits above the pin, which moves out of the groove of the rod due to the pressure of the gas spring. The rod is therefore released and is able to move into contact with a plunger in the drug cartridge due to the gas pressure and transmits this pressure onwards so that the product is administered from the cartridge.

With the known locking systems for administering devices, it is often not possible to unlock the device and then return it to a locked state without proceeding with the administration procedure or effecting some other movement of the device. Furthermore, in the case of most administering devices, reversing the unlocking or subsequent administration is effected by a movement in the direction in which the product is administered. The user immediately associates such a movement in the administering direction with the idea that an administration will follow directly. With known administering devices, it is often not possible to avoid exerting a releasing pressure in addition to the pressure needed to hold the device on the tissue surface in the first place.

For patients who find administration extremely unpleasant, it is advantageous if the unlocking and administering procedures are based on movements which are not in the direction of administration. Additional pressure on the tissue surface can be avoided as a result. It is also advantageous if an unlocked administering device can be returned to a locked state or relocked without having to proceed with an administration, e.g., if an incorrect time was chosen for the administration.

### SUMMARY

An object of the present invention is to provide a device for administering an injectable product, which comprises a locking mechanism that is reliable and easy to operate, enabling a locked or unlocked state to be obtained at any time, and which facilitates the administering procedure and permits a simple release.

In one embodiment, the present invention comprises a device for administering an injectable product comprising a drive unit for driving a drive member in the direction of a longitudinal axis of the device, a release mechanism for releasing the drive unit, a locking mechanism which prevents operation of the release mechanism when locked, wherein the locking mechanism can be displaced along the longitudinal axis of the device into an unlocked position and the release mechanism can be displaced radially with respect to the longitudinal axis for releasing purposes when the locking mechanism is in the unlocked position.

In one embodiment, the present invention comprises a device for administering an injectable product, the device having a longitudinal axis and comprising a drive unit, a release for releasing the drive unit, and a lock for the release, wherein the lock is moveable generally along or parallel to the longitudinal axis into an unlocked position and wherein the release is moveable generally radially with respect to the longitudinal axis to release the drive unit when the lock is in the unlocked position.

In one embodiment, the present invention comprises a device for administering an injectable product comprising a drive unit for driving a movement of a drive member in the direction of a longitudinal axis of the device, a release mechanism for releasing the drive unit, and a locking mechanism, which, in the locked position, prevents the release mechanism from being operated. The administering device has an elongate housing, in which the drive unit and a product container, such as an ampoule, are accommodated. The drive unit may be provided in the form of a conventional spring mechanism whereby a dispensing mechanism with a plunger rod is driven by a helical spring in the direction towards a plunger in the ampoule. The plunger then forces the fluid product in the ampoule towards an oppositely lying outlet so that it is dispensed. However, the invention may also be employed with a device designed for a needleless administration procedure. Furthermore, the spring mechanism may also act on a forward-moving element of a piercing mechanism for piercing an injection needle through a tissue surface so that when the drive unit is released, the injection needle automatically pierces the surface. Naturally, any suitable drive mechanism could be used for or in the drive unit.

In one embodiment, the locking mechanism for blocking or preventing operation of the release mechanism prior to operation is initially disposed in a locking position, from which it can be moved along the longitudinal axis of the device into an unlocked position in which the release mechanism can be operated. The movement whereby the administering device is unlocked is effected in the direction opposite a dispensing direction in which the product is dispensed from the administering device and opposite the driving direction of the drive member. On unlocking the device, the user therefore does not associate this with an unlocking procedure which takes place just before an administering procedure.

When the locking mechanism is in the unlocked position, the release mechanism is released by it so that the release mechanism is able to move radially with respect to the longitudinal axis. The release mechanism can then be manually operated and the drive unit released or freed. Due to the fact that the release mechanism is operated radially, in some preferred embodiments by pushing laterally into the device perpendicular to the longitudinal axis, no additional pressure occurs in the direction of the longitudinal axis of the administering device.

In a preferred embodiment of the present invention, the release mechanism is provided in the form of a longitudinally extending release plate, which projects out from the administering device by one of its ends, while the other end lies in the interior of the device and which, in a central region, has an orifice with a narrow and a wide portion. The drive member of the drive unit is generally rod-shaped, oriented in the longitudinal direction of the administering device and has a head region at one end incorporating a region with a smaller diameter than in adjacent regions. The drive member co-operates with the release plate so that the region with the small diameter fits positively in the narrow orifice portion, thereby preventing the drive member from effecting any translating movement relative to the release plate along the longitudinal axis of the device. In a locked position, therefore, the drive member of the drive unit is retained by the release mechanism in a fixed position relative to the housing of the administering device. The drive unit is then already pre-tensioned.

In order to release the drive unit, the release plate can be pushed radially inwards with respect to the longitudinal axis, into the housing of the administering device, to a position in which the locking mechanism is unlocked. As a result, the drive member is pushed inside the orifice in the release plate from the narrow portion to the wide portion because the drive member is held stationary in the radial direction by the housing of the administering device. The wide portion of the orifice in the release plate is dimensioned so that the area of the head region of the drive member with the bigger diameter fits through this portion and in this position, the drive member is able to move relative to the release plate in the longitudinal direction. When the release plate is pushed inwards in the radial direction, therefore, the drive member of the drive unit is released and effects a movement in the direction of the longitudinal axis of the device due to the pre-tensioning of the drive unit. Consequently, the piercing mechanism for effecting the piercing movement of an injection needle or the dispensing mechanism for dispensing the injectable product can be driven. Advantageously, the piercing mechanism which inserts the injection needle in a tissue surface is driven first of all and, directly after which, the dispensing mechanism for dispensing the injectable product through the injection needle into the tissue is driven.

In a preferred embodiment of the invention, the locking mechanism is provided in the form of a sleeve, disposed in the region of the release plate around the housing of the administering device, preferably at an end of the device lying opposite a needle unit. The sleeve may also form a part of the housing. In the region of the release plate, the sleeve has an orifice through which the plate can extend. In the longitudinal direction of the administering device, the orifice is so wide that the sleeve can be moved in the longitudinal direction of the administering device in spite of the fact that the release plate extends through the orifice. At its outwardly lying end, the release plate has a projection extending perpendicular to the plate and parallel with the housing wall. In order to lock the release plate in the position in which the plate fixes the drive member, a sleeve wall engages between the projection and the housing wall in the longitudinal direction, preferably in the administering direction. Due to the fact that the sleeve wall is engaged, the release plate is prevented from moving in the radial direction with respect to the longitudinal axis, so that the locking mechanism is blocked and is disposed in a locking position. The projection may also be provided in the form of a recess in the plate.

In order to unlock the administering device, the sleeve is pushed in the longitudinal direction until the sleeve wall has been pulled out of engagement between the plate projection and housing wall and the projection releases the release plate. The sleeve orifice then abuts with the release mechanism by means of a face lying opposite the engagement side. The release plate is therefore no longer prevented from moving radially by the sleeve and can thus move in the radial direction in the housing and the sleeve can be pushed inwards. However, it is also possible to push the sleeve back in the opposite direction and bring the sleeve wall back into engagement between the plate projection and housing wall so that the locking mechanism is returned to a locked position. In some preferred embodiments, the sleeve is guided on the housing and with respect to the housing by means of guide mechanisms. It is also of advantage to provide a catch mechanism in which the locking mechanism latches in the locked position, so that the sleeve can be reliably retained in this position. In order to displace the sleeve, a slight initial resistance then has to be overcome before the sleeve can be pushed into an unlocked position.

In one preferred embodiment, the administering device is designed as a re-usable device. To this end, after administering a product, the drive unit can be returned to a pre-tensioned state and the drive member secured by the release mechanism again. The drive unit can be re-set when a new ampoule is inserted, for example. This being the case, the head region of the drive member is guided back through the orifice of the release plate and the release plate is returned, by means of a spring for example, to the initial position in which the region with the small diameter of the head region fits in the narrow portion of the plate orifice.

In another embodiment of the present invention, in order to provide an additional lock for the release mechanism, the locking mechanism also secures a needle protector in a position protecting the needle. The needle protector is provided in the form of a longitudinally extending sleeve, for example, which projects beyond the injection needle by means of its front end in a first pushed-forward position, extending along the longitudinal axis of the device as far as the region on which the locking mechanism is provided. In the locked state, the sleeve of the locking mechanism is in a position in which it is pushed forward with respect to the administering device, i.e., pushed onto the housing. In this position, it acts as a stop for the needle protector sleeve so that the latter can not be moved along the longitudinal axis of the administering device in the direction opposite the administering direction into a second retracted position. When the locking mechanism is moved into an unlocked position, i.e., is pushed along the longitudinal axis in the direction opposite the administering direction, the stop moves away from the needle protector sleeve. The needle protector sleeve can then be moved into the unlocked position by the same distance as the locking sleeve in the direction opposite the administering direction and, when placed on a tissue surface for example, can be moved into the second retracted position.

It is also possible to design the needle protector sleeve with another locking function for the release mechanism. To this end, the needle protector sleeve is extended in the interior of the locking sleeve, at least in the region lying opposite the end of the release mechanism and opening into the housing. When the release mechanism is in an initial position, it sits against the wall of the needle sleeve in a locking position of the needle protector sleeve and the locking sleeve. The needle sleeve can not be displaced and retracted until the locking sleeve is pushed into an unlocked position. When the needle sleeve is in the retracted state, an orifice in the needle sleeve lies opposite the release mechanism, through which the release mechanism can pass when it is pushed radially inwards in order to release the drive unit.

An administering device in accordance with the present invention affords optimal possible protection against inadvertent operation of the device and against injury due to piercing by the needle. Since both the locking mechanism and the release mechanism do not have to be operated in the direction of an administering procedure, it is easier for a user to overcome the inhibiting threshold needed in order to initiate an administering process. In addition, not only is the needle protected or covered to prevent access at any time, the needle protector projects far beyond the needle and also provides visual protection, which also makes the administering device suitable for users who have a needle phobia. The administering device is easy to use because, once unlocked, a pressure merely has to be applied to the release mechanism in order to proceed with administering the product, without the need for further manipulations.

### BRIEF DESCRIPTION OF THE DRAWINGS

FIG. 1 is a longitudinal section through one embodiment of an administering device in accordance with the present invention, in a locked position,

FIG. 2 is a longitudinal section of the device illustrated in FIG. 1, in an unlocked position,

FIG. 3 is a longitudinal section of the device illustrated in FIG. 1, in a released state,

FIG. 4 is a longitudinal section through the device illustrated in FIG. 1, after an administering procedure,

FIG. 5 is a longitudinal section through another embodiment of an administering device in accordance with the present invention, in a locked position, and

FIG. 6 is a longitudinal section of the device illustrated in FIG. 5, after an administering procedure.

### DETAILED DESCRIPTION OF THE DRAWINGS

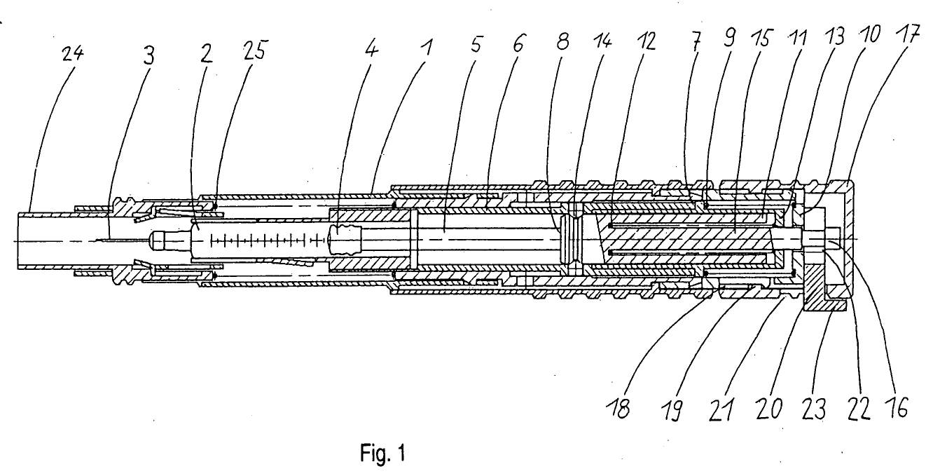

FIG. 1 illustrates an embodiment of an administering device in accordance with the present invention in the exemplary form of an automatic injection pen. The injection pen has an elongate housing 1, in which an ampoule 2 containing an injectable product or substance is accommodated. At one end, which can be thought of as the administering end, the device carries an interchangeable injection needle 3. Housed inside the ampoule 2 at an end lying opposite the injection needle is a plunger 4, which closes off the ampoule 2. Adjoining the plunger 4 is a plunger rod 5 extending longitudinally in the injection pen. The unit comprising the injection needle 3, ampoule 2, plunger 4 and plunger rod 5 is fixedly mounted in a piercing sleeve 6, which is displaceable in the longitudinal direction relative to the housing 1, being illustrated in the position retracted into the housing in FIG. 1, secured by a low catch resistance for example. The piercing sleeve 6 is connected via a stop 7 to a drive unit, by which the piercing sleeve 6 can be displaced in an administering direction, i.e., toward the needle and in the direction in which the injection needle 3 points, along or parallel to the longitudinal axis of the injection pen, so that the injection needle 3 is automatically moved or driven to pierce a tissue surface by the drive unit.

The plunger rod 5 is likewise connected to the drive unit by a stop 8 so that it can push the plunger 4 in the administering direction along the longitudinal axis inside the ampoule 4 to administer the injectable product.

To drive the piercing sleeve 6, the drive unit has a first spring 9 inserted between the stop 7 for the piercing sleeve 6 and a rear housing wall 10 extending perpendicular to the longitudinal axis of the injection pen. The spring 9 is illustrated in a pre-tensioned, compressed state in FIG. 1. Disposed inside the piercing sleeve 6 is a dispensing sleeve 11, which adjoins the plunger rod 5 and is displaceable relative to the piercing sleeve 6 and housing 1 in the longitudinal direction of the injection pen. Tto drive the dispensing sleeve 11, the drive unit has a second spring 12, which is inserted between a wall 13 closing off the piercing sleeve 6 and a stop on the dispensing sleeve 11. In FIG. 1, the second spring 12 is illustrated in a pre-tensioned, compressed state. The dispensing sleeve 11 is retained inside the piercing sleeve 6, e.g., by means of a resistance 14, in the pre-tensioned position until the piercing procedure has been completed. The piercing and dispensing mechanism illustrated here constitutes the subject matter of a parallel application owned by the owner of the present application and is included herein by reference.

Accordingly, the drive unit incorporates at least the piercing sleeve 6 and the dispensing sleeve 11, which are displaceable in the longitudinal direction of the injection pen, as well as the first spring 9 and the second spring 12.

Provided on the dispensing sleeve 11 is a rod-type extension 15, which, in some embodiments, preferably starts at a front end of the dispensing sleeve 11, extends through and along the axis of the dispensing sleeve 11 and extends out from the dispensing sleeve 11 via the rear end. The end of the extension 15 extending out from the dispensing sleeve 11 projects through an orifice in the wall 13 and an orifice in the wall 10 and at this end has a head region 16 with a region having a smaller diameter than at the adjacent regions.

Provided generally at the end of the injection pen from which the extension 15 projects is a sleeve cap 17 serving as a locking mechanism. The sleeve cap 17 is displaceable relative to the injection pen in the longitudinal direction and can form a housing closure for the injection pen. In order to guide the sleeve cap 17, guide mechanisms are provided in the form of guide grooves 18 on the housing 1 and inwardly directed guide projections 19 on the sleeve cap 17 engaging in the guide grooves 18. The guide mechanism may simultaneously provide a catch mechanism, which retains the sleeve cap 17 in the pushed-in position illustrated in FIG. 1, i.e., in a foremost position in the administering direction.

In terms of a release mechanism for releasing the drive unit, the injection pen has an elongate release plate 20 which is disposed perpendicularly to the longitudinal direction of the injection pen and extends out through an orifice 21 in the sleeve cap 17 with one end, protruding out from the side of the injection pen. The other end of the release plate 20 opens into the interior of the housing 1. In the region of the release plate lying in the interior of the pen, it has an orifice 22 with a narrow orifice portion, the width of which corresponds to the small diameter of the head region 16 of the extension 15, and a wide orifice portion, the width of which corresponds to the bigger diameter of the head region 16 of the extension.

As illustrated in FIG. 1, the head region 16 of the extension 15 projects through the orifice 22 of the release plate 20 and the small diameter of the head region 16 lies in a positive fit in the narrow orifice portion of the orifice 22 so that the regions of the head region 16 with the bigger diameter bordering the region with the small diameter form an abutment of the extension 15 on the release plate 20 and the extension 15 is not able to move inside the orifice 22. The drive unit is therefore retained in a state in which it is pre-tensioned by the release plate 20 due to the compressed spring.

At the end protruding out from the side of the injection pen, the release plate 20 has a projection in the form of a lug 23, which stands vertically out of the release plate 20 in the longitudinal direction of the injection pen in the direction opposite the administering direction. The release plate 20 therefore extends sufficiently far out of the sleeve cap 17 through the orifice 21 that an internal wall of the lug 23 lies on and parallel with an external wall of the sleeve cap 17. The distance between the lug internal wall, i.e., the lug wall facing the housing, and the orifice wall of the orifice 22 terminating the narrow orifice portion of the orifice lying farthest away from the lug internal wall, is dimensioned so that the release plate 20 is not able to move in the radial direction with respect to the longitudinal axis as long as the lug internal wall lies against the external wall of the sleeve cap 17 on the lug wall. The release plate 20 is therefore not able to move in the radial direction and can therefore not effect a relative movement with respect to the extension 15. In this state, illustrated in FIG. 1, the sleeve cap 17 is retained in a locked state for the injection pen and is retained therein, e.g., by the catch mechanism in the guide mechanism. This prevents the injection pen from being undesirably and inadvertently released.

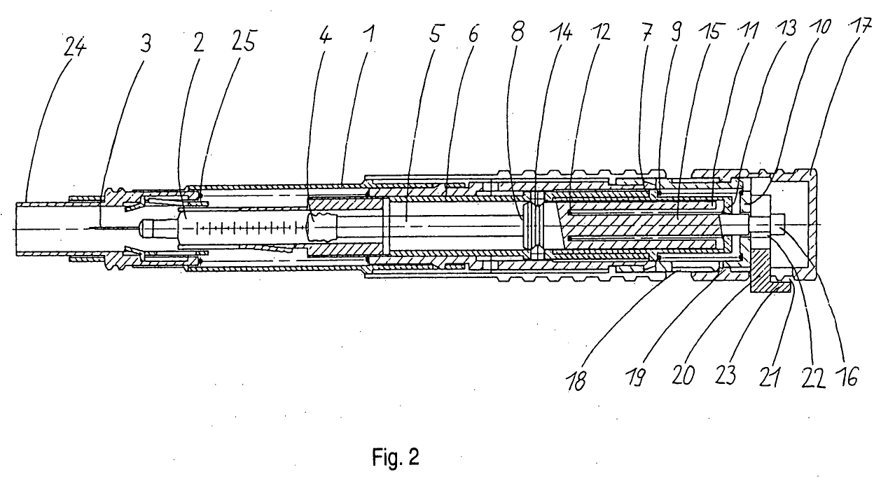

FIG. 2 illustrates the embodiment of the injection pen illustrated in FIG. 1 in an unlocked position. In order to unlock the injection pen, the sleeve cap 17 has been pulled back from the injection pen, i.e., in the direction opposite the administering direction or away from the needle, generally along the longitudinal axis of the pen inside the guide mechanism, into a rear position as far away from the injection needle as possible. The length of the orifice 21 of the sleeve cap 17 in the longitudinal direction corresponds to the length of the lug 23, i.e., to the thickness of the release plate 20 plus the length of the projecting lug. When the sleeve cap 17 is pulled out in order to unlock the injection pen, the cap 17 can be pushed in the direction opposite the administering direction on the housing 1 until the orifice 21 sits against the release plate 20 and does so on the side lying opposite the lug 23. Accordingly, the wall of the sleeve cap 17 which was lying opposite the lug 23 is also moved as well. The lug wall therefore no longer lies opposite a wall region of the sleeve cap 17, but is opposite an orifice region of the orifice 21. The lock preventing the radial movement of the release plate 20 afforded by the sleeve cap 17, illustrated in FIG. 1, no longer exists in FIG. 2 because the sleeve cap 17 has been pulled out so that the injection pen is in an unlocked position. In accordance with the present invention, it is advantageous if the sleeve cap 17 can also be pushed back into a forward position relative to the housing 1, without operating the release plate 20, in which it blocks any movement of the release plate 20 again and thus resumes a locking position.

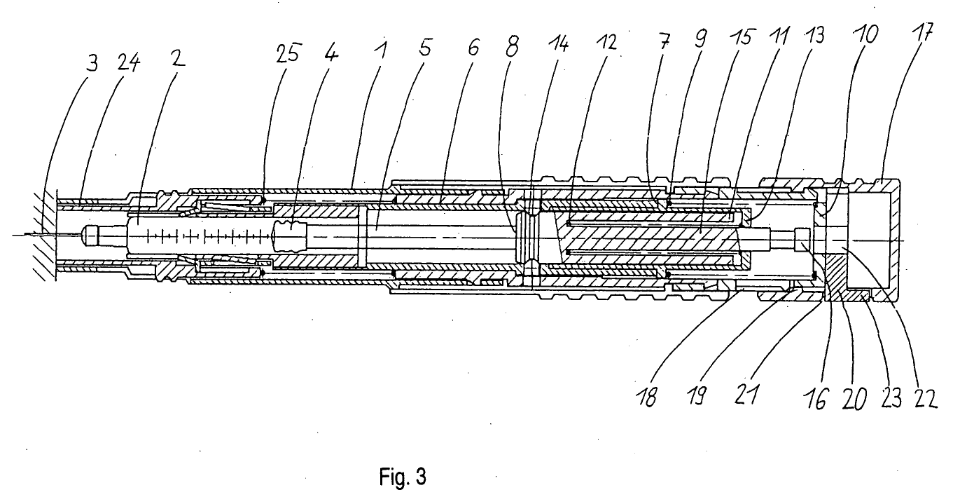

FIG. 3 shows the injection pen in a released position, in which the release plate 20 was moved in the radial direction towards the generally central longitudinal axis of the injection pen. To this end, the release plate 20 is pushed manually, e.g., with the thumb of one hand, into the sleeve cap 17, as a result of which the extension 15 moves inside the orifice 21 from the narrow orifice portion into the wide orifice portion. The wide orifice portion of the orifice 21 is sufficiently wide that the regions of the head region 16 of the extension 15 with a relatively larger diameter are also able to fit through the orifice 21 and the extension is therefore able to move inside the orifice 21 and out of it. Once the release plate 20 has been pushed in, it, together with its lug 23, is moved so that it lies inside the orifice 21 of the sleeve cap 17. The sleeve cap 17 can then no longer be moved into a forward inserted position.

The inward pushing action of the release plate 20 causes the drive unit to be released and a piercing and dispensing procedure takes place due to the pre-tensioned springs 9 and 12. As soon as the positive co-operation between the release plate 20 and the extension 15 no longer exists because the release plate has been pushed in, the piercing sleeve 6 is firstly pushed relative to the housing 1 in the administering direction by the force of the first spring 9 so that the injection needle 3 is moved in the direction of the tissue surface.

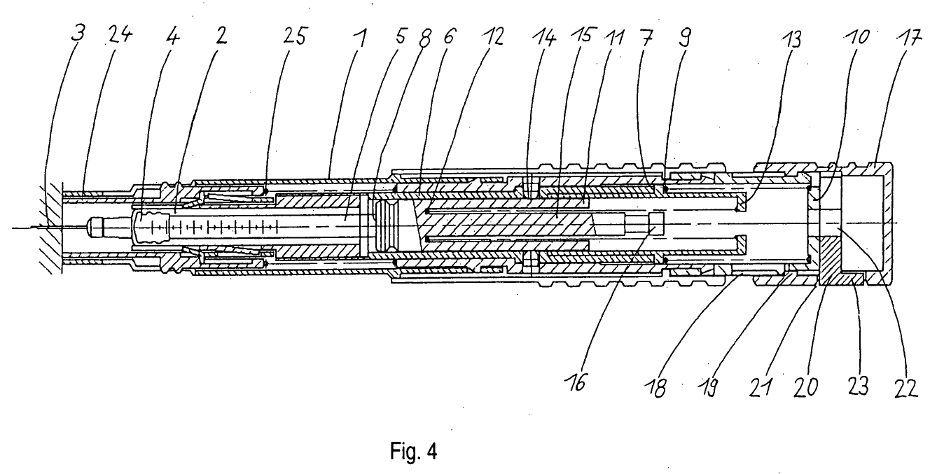

Due to the force of the second spring 12, the dispensing sleeve 11 is then likewise moved relative to the housing 1 and the piercing sleeve 6 moves in the administering direction so that the plunger 4 inside the ampoule 2 is pushed in the direction of the injection needle, causing dispensing through the inserted needle, as illustrated in FIG. 4.

In brief summary, after the injection needle 3 has first pierced a tissue surface, the product in the ampoule 2 is administered. It should be appreciated that the springs 9 and 12 and the resistances provided between housing 1, the piercing sleeve 6 and the dispensing sleeve 11 are and may be adapted with respect to one another to guarantee a correct sequence and force during administration.

As illustrated in FIGS. 3 and 4, it is possible, in accordance with one or more embodiments of the present invention, to provide a needle protector sleeve 24 which is displaceable relative to the housing 1 and extends far beyond the housing 1 so that it also surrounds the injection needle 3 in a pushed-forward state. In order to administer the product, the injection pen incorporating the needle protector sleeve 24 is pressed generally perpendicularly onto a tissue surface and the needle protector sleeve 24 is pushed back into the interior of the housing 1 until an edge of the housing 1 lies on the surface. As the injection needle 3 effects the piercing action by means of the drive unit, the injection needle 3 moves out beyond the edge of the housing 1 and needle protector sleeve 24 in the administering direction and pierces the tissue surface.

After the administering procedure, the injection pen is removed from the tissue surface and the injection needle 3 pulled out from the tissue surface. At the same time, the needle protector sleeve 24 is pushed back into a position projecting beyond the housing 1 by a spring 25. The spring 25 is inserted between the needle protector sleeve 24 and a stop on the housing 1. The injection needle 3 is therefore protected by the needle protector sleeve 24 at all times, even if it is in a position pushed forward inside the injection pen.

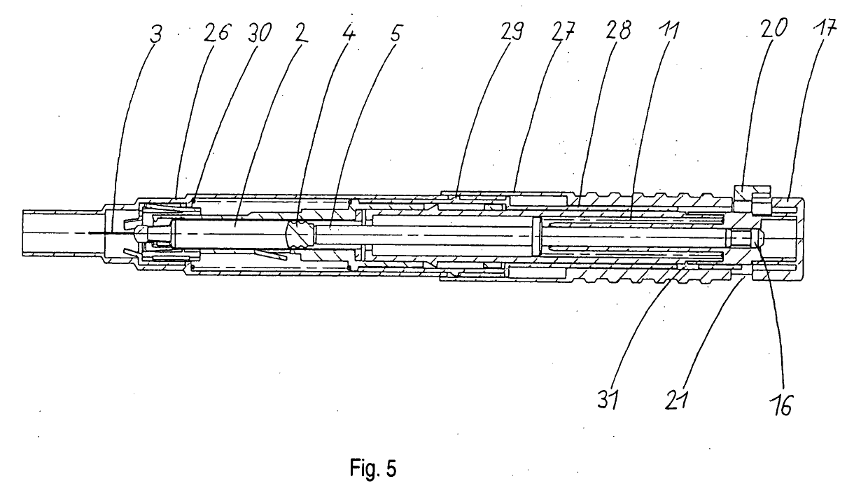

FIG. 5 illustrates a second embodiment of an administering device in accordance with the present invention, also in the form of an injection pen. The locking mechanism operates in the same way as that of the embodiment illustrated in FIGS. 1 to 4, i.e., by means of the sleeve cap 17 and the release plate 20. In addition, however, a further locking mechanism is provided, which is intended to ensure that release does not occur until a needle protector is in a retracted state, i.e., the injection pen has been correctly placed on a tissue surface. To this end, the needle protector has a front needle sleeve 26 forming a part of the housing displaceable relative to a base housing 27, and a locking sleeve 28 displaceable relative to the needle sleeve 26 and to the base housing 27. The sleeve cap 17 can be moved relative to the base housing 27 to lock the injection pen. Provided on the locking sleeve 28 are stops 29, against which a rear end of the needle sleeve 26 abuts so that when the needle sleeve 26 moves relative to the base housing 27 in the direction opposite the administering direction, it moves the sleeve 28 with it during this movement. Accordingly, a spring 30 compresses. The locking sleeve 28 projects with its end lying opposite the needle sleeve 26 through the base housing 27 and along the internal face into the sleeve cap 17 to beyond the region lying opposite the release plate 20. The end of the release plate 20 opening into the sleeve cap 17 is moved so that it lies opposite the internal face of the locking sleeve 28 when the injection pen is in a locked position, i.e., the needle sleeve 26 and the locking sleeve 28 are in a position extended from the base housing 27. The end of the locking sleeve 28 projecting into the sleeve cap 17 has an orifice 31, the diameter of which corresponds to the width of the release plate 20.

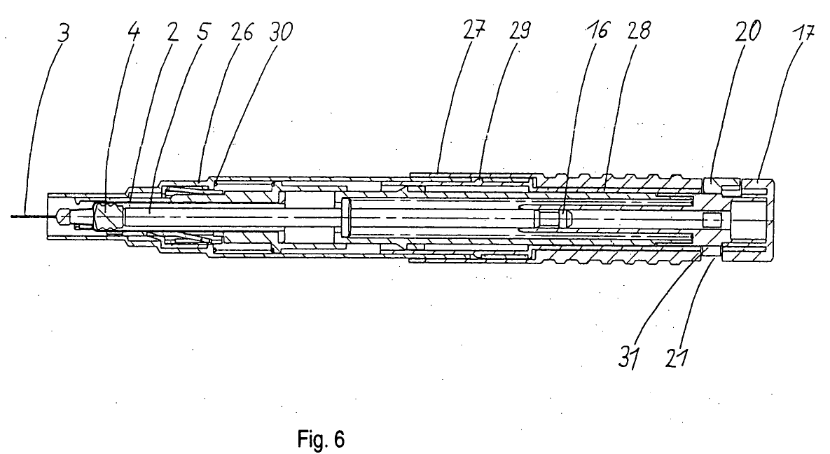

As illustrated in FIG. 6, when the injection pen is placed on a tissue surface, the needle sleeve 26 and the locking sleeve 28 are pushed into the base housing 27 until the orifice 31 of the locking sleeve 28 lies opposite the release plate 20. Once the sleeve cap 17 has been pulled out, the release plate 20 can be pushed inwardly in the radial direction, as in the exemplary embodiment illustrated in FIGS. 1 to 4, as a result of which the end of the release plate 20 disposed in the sleeve cap 17 moves through the orifice 31 of the locking sleeve 28 and the extension 15 moves out of the orifice 22. Consequently, the drive unit is released and piercing can proceed as the injection needle 3 moves out beyond the front edge of the needle sleeve 26.

When the injection pen is removed from the tissue surface, i.e., as the injection needle 3 is pulled out of the tissue surface, the needle sleeve 26 is moved back in the administering direction (toward the piercing needle tip) by the spring 30 into a pushed-forward position in which it surrounds the forwardly positioned injection needle 3. As this happens, the locking sleeve 28 remains stationary with respect to the base housing so that the needle sleeve 26 is also displaced relative to the locking sleeve 28.

As may be appreciated from the embodiments of the present invention illustrated and described as examples, various modifications may be made to the administering devices without departing from the scope of the invention, provided suitable locking and release mechanisms or structures are provided.

Embodiments of the present invention, including preferred embodiments, have been presented for the purpose of illustration and description. They are not intended to be exhaustive or to limit the invention to the precise forms and steps disclosed. Obvious modifications or variations are possible in light of the above teachings. The embodiments were chosen and described to provide the best illustration of the principles of the invention and its practical application, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. A device for administering an injectable product, the device having a longitudinal axis and comprising a drive unit for driving a drive member in the direction of the longitudinal axis, a release mechanism for releasing the drive unit, a locking mechanism having a locked and an unlocked position, which when in the locked position prevents operation of the release mechanism, wherein the locking mechanism is displaceable generally along the longitudinal axis into an unlocked position and the release mechanism displaceable generally radially with respect to the longitudinal axis for releasing purposes when the locking mechanism is in the unlocked position.

2. The administering device as claimed in claim 1, wherein the locking mechanism can be displaced in a direction opposite to a dispensing direction of the injectable product into the unlocked position.

3. The administering device as claimed in claim 1, wherein the release mechanism can be manually operated.

4. The administering device as claimed in claim 1, wherein the locking mechanism can be returned to the locked position without operating the release mechanism.

5. The administering device as claimed in claim 1, wherein the release mechanism can be re-set.

6. The administering device as claimed in claim 1, wherein the drive unit further comprises at least one of a piercing mechanism for causing an injection needle to pierce a surface and a dispensing mechanism for dispensing the injectable product from a product container.

7. The administering device as claimed in claim 6, wherein the drive unit drives the piercing mechanism and the dispensing mechanism consecutively.

8. The administering device as claimed in claim 1, further comprising a needle protecting mechanism, wherein, when the locking mechanism is in the locked position, the needle protector mechanism is locked in a protecting position.

9. The administering device as claimed in claim 1, further comprising a needle protector mechanism moveable between a needle-protecting position and a retracted position, wherein when the needle protecting mechanism is in the needle-protecting position the release mechanism is locked, and when the needle protecting mechanism is in the retracted position the release mechanism is unlocked.

10. A device for administering an injectable product, the device having a longitudinal axis and comprising a drive unit, a release for releasing the drive unit, and a lock for the release, wherein the lock is moveable along or parallel to the longitudinal axis into an unlocked position and wherein the release is moveable radially with respect to the longitudinal axis to release the drive unit when the lock is in the unlocked position.

11. The device according to claim 10, wherein the drive unit comprises a piercing sleeve, a dispensing sleeve, a first spring and a second spring.

12. The device according to claim 11, wherein the lock comprises a sleeve cap.

13. The device according to claim 12, wherein the release comprises a plate having two ends, the plate arranged generally perpendicularly to the longitudinal axis with one end extending through the sleeve cap and the other end inside the device.
